(19) United States
(12) Patent Application Publication
(10) Pub. No.: US 2023/0301927 A1
(43) Pub. Date: Sep. 28, 2023

(54) PROCESS FOR PRODUCING AN ORALLY ADMINISTERED PHARMACEUTICAL COMPOSITION WITH COLONIC DELIVERY

(71) Applicants: ETHYPHARM, SAINT-CLOUD (FR); Shanghai Ethypharm Pharmaceuticals Co. Ltd., SHANGHAI (CN)

(72) Inventors: Catherine HERRY, SAINT-OUEN DU TILLEUL (FR); Yuqiu ZHU, SHANGHAI (CN); Bruno CRIERE, GRAVIGNY (FR)

(21) Appl. No.: 17/776,723

(22) PCT Filed: Nov. 13, 2020
(86) PCT No.: PCT/FR2020/052082

§ 371 Date: Oct. 26, 2022

(30) Foreign Application Priority Data
Nov. 13, 2019 (CN) 201911103911.6
Nov. 13, 2019 (FR) FR1912643

(51) Int. Cl. A61K 9/50; A61K 47/32; A61K 31/606

(52) U.S. Cl. CPC A61K 9/5089; A61K 47/32; A61K 31/606

(57) ABSTRACT

The present invention relates to a process for preparing an orally administered pharmaceutical composition with colonic delivery, comprising at least one core and a coating layer, making it possible to obtain a pharmaceutical composition which exhibits uniform and reproducible dissolution and therefore likewise uniform and reproducible release of the active ingredient with low coefficients of variation, said process being characterized in that it comprises the following steps:
a) Spraying onto a neutral support an aqueous suspension comprising at least one anionic (meth)acrylate copolymer that is soluble at a pH greater than 5.5 and at least one active ingredient intended to be delivered in the colon;
or
a′) Spraying onto a neutral support an aqueous suspension comprising at least one anionic (meth)acrylate copolymer that is soluble at a pH greater than 5.5 then
b′) Dusting at least one active ingredient intended to be delivered in the colon onto the microgranules obtained after step a′);
c′) carrying out steps a′) and b′) alternately until the desired content of active ingredient has been obtained
and
d) Coating the microgranules obtained after step a) or c′) by spraying a composition comprising at least one anionic (meth)acrylate copolymer that is soluble at a pH greater than 6, an anionic (meth)acrylate copolymer that is soluble at a pH greater than 7 and an anionic (meth)acrylate copolymer that is insoluble in an aqueous medium.

### FIELD OF THE INVENTION

The present invention relates to a process for preparing an orally administered pharmaceutical composition with colonic delivery comprising at least one core and a coating layer.

### PRIOR ART

Mesalazine or 5-aminosalicylic acid is an anti-inflammatory commonly used for the treatment of inflammatory bowel diseases such as ulcerative colitis or Crohn's disease.

Mesalazine acts locally in the colon. However, the oral administration of mesalazine is made problematic by the fact that this drug is almost completely absorbed in the small intestine and that, consequently, only a small amount reaches the colon to ensure its therapeutic action.

In the past, with the aim of overcoming this difficulty, mesalazine formulations with particular coatings have been developed, having the characteristic of releasing the active ingredient only in the desired area, as well as to avoid systemic side effects. These pharmaceutical compositions are delayed or slow release forms, suitable for preventing or delaying the absorption of mesalazine in the proximal tract in order to obtain therapeutic concentrations in the ileum and the colon.

For example, European patent application EP 0 040 590 (applicant Aktiebolaget Hässle) describes oral pharmaceutical preparations capable of releasing a drug, for example mesalazine, selectively in the colon, at a pH greater than 5.5. This is obtained by coating a core containing the active ingredient with a mixture of an anionic acrylic polymer soluble just at pH 5.5, such as, for example, Eudragit L, in amounts ranging from 10 to 85%, and an acrylic polymer substituted with quaternary ammonium, insoluble in water, such as for example Eudragit RS or RL, in amounts ranging from 15 to 90%. These compositions are first produced by producing the core by mixing the active ingredient with a filler material then extrusion/spheronization or by depositing the active ingredient on the surface of a support particle or by producing a core containing only the active ingredient; then, the core is coated and this application of the coating is preferably carried out using a fluidized bed apparatus. However, the compositions described in this document do not allow to obtain a uniform and reproducible coating, which leads to a dissolution of the composition and a non-uniform release of the active ingredient in the intestinal tract. Indeed, the production of microgranules by extrusion/spheronization leads to obtaining microgranules that are heterogeneous in size; thus, during coating, these microgranules will receive a different amount of polymer depending on their size, which will form a heterogeneous polymeric layer. The impact of this polymeric layer heterogeneity is subsequently reflected by a variability in dissolution between the coated microgranules.

Alternatives were then sought, aiming at acting on the transit time from the mouth to the terminal ileum of the produced pharmaceutical composition or on the enzymatic activity of the colonic microbial flora.

Nevertheless, there is still a need to have a process for producing an orally administered pharmaceutical composition with colonic release allowing the release of the active ingredient of the composition obtained to be uniform and targeted in the colon and which is simple, reproducible and economical.

### SUMMARY OF THE INVENTION

In the context of the present invention, the inventors have discovered a process for preparing a pharmaceutical composition which allows to release an active ingredient, in particular mesalazine, in the colon, in a specific and uniform manner. This process involves a technique of dusting the active ingredient which allows to guarantee a uniform and reproducible particle size and coating with low coefficients of variation, and therefore likewise uniform and reproducible release of the active ingredient with low coefficients of variation.

The present invention thus relates to a process for preparing an orally administered pharmaceutical composition with colonic delivery comprising at least one core and a coating layer, characterized in that it comprises the following steps:
a) Spraying onto a neutral support an aqueous suspension comprising at least one anionic (meth)acrylate copolymer that is soluble at a pH greater than 5.5 and at least one active ingredient intended to be delivered in the colon;
or
a′) Spraying onto a neutral support an aqueous suspension comprising at least one anionic (meth)acrylate copolymer that is soluble at a pH greater than 5.5 then
b′) Dusting at least one active ingredient intended to be delivered in the colon onto the microgranules obtained after step a′);
c′) carrying out steps a′) and b′) alternately until the desired content of active ingredient has been obtained
and
d) Coating the microgranules obtained after step a) or c′) by spraying a composition comprising at least one anionic (meth)acrylate copolymer that is soluble at a pH greater than 6, an anionic (meth)acrylate copolymer that is soluble at a pH greater than 7 and an anionic (meth)acrylate copolymer that is insoluble in an aqueous medium.

### DETAILED DESCRIPTION OF THE INVENTION

### Definitions

By “colonic delivery” or “colonic release” is meant, within the meaning of the present invention, the selective release of an active ingredient in the colon, also called the large intestine.

By “uniform” release or dissolution is meant, within the meaning of the present invention, a uniform dissolution between the microgranules of a pharmaceutical composition, in terms of dissolution time and of the area of the intestinal tract in which they release the active ingredient that they contain. Uniform dissolution aims at obtaining the same kinetics of release of the active ingredient in the body between the different microgranules. In the context of the present invention, the uniform dissolution aims at allowing a localized release of active ingredient in the colon by the majority of the microgranules of the composition according to the invention, allowing on the one hand a greater concentration of active ingredient at the therapeutic target area which is the colon and on the other hand to avoid the side effects related to the release of active ingredient in the other regions of the intestinal tract and to the absorption of the active ingredient in the small intestine.

By “specific” or “targeted” release or dissolution is meant, within the meaning of the present invention, the fact that the release of the active ingredient is specific or targeted to the part of the intestine considered relative to the other regions of the intestinal tract.

By “anionic copolymer” is meant, within the meaning of the present invention, a copolymer containing anionic groups.

By “(meth)acrylate copolymer” is meant, within the meaning of the present invention, a copolymer obtained by polymerization of (meth)acrylate monomers such as acrylic acid, methacrylic acid or esters thereof. The (meth)acrylate copolymer can in particular be a polymer resulting from the polymerization of at least two of the following monomers: acrylic acid, methacrylic acid, ethyl acrylate, methyl methacrylate, and methacrylic acid ester with a quaternary ammonium group.

By “water-insoluble copolymer” is meant, within the meaning of the present invention, any copolymer that is insoluble in water or in a physiological solution.

By “D50” is meant, within the meaning of the present invention, the diameter that 50% of the considered population of microgranules have.

By “D90” is meant, within the meaning of the present invention, the diameter that 90% of the considered population of microgranules have.

Process for Preparing a Pharmaceutical Composition According to the Invention

The present invention relates to a process for preparing an orally administered pharmaceutical composition with colonic delivery comprising at least one core and a coating layer, characterized in that it comprises the following steps:
a) Spraying onto a neutral support an aqueous suspension comprising at least one anionic (meth)acrylate copolymer that is soluble at a pH greater than 5.5 and at least one active ingredient intended to be delivered in the colon;
or
a′) Spraying onto a neutral support an aqueous suspension comprising at least one anionic (meth)acrylate copolymer that is soluble at a pH greater than 5.5 then
b′) Dusting at least one active ingredient intended to be delivered in the colon onto the microgranules obtained after step a′);
c′) carrying out steps a′) and b′) alternately until the desired content of active ingredient has been obtained
and
d) Coating the microgranules obtained after step a) or c′) by spraying a composition comprising at least one anionic (meth)acrylate copolymer that is soluble at a pH greater than 6, an anionic (meth)acrylate copolymer that is soluble at a pH greater than 7 and an anionic (meth)acrylate copolymer that is insoluble in an aqueous medium.

This process therefore allows to obtain an orally administered pharmaceutical composition with colonic delivery comprising at least one core and a coating layer, said core comprising at least one neutral support, an active ingredient intended to be delivered in the colon and at least one anionic (meth)acrylate copolymer that is soluble at a pH greater than 5.5, said coating layer comprising at least one anionic (meth)acrylate copolymer that is soluble at a pH greater than 6, an anionic (meth)acrylate copolymer that is soluble at a pH greater than 7 and a water-insoluble anionic (meth)acrylate copolymer.

According to a preferred embodiment, the orally administered pharmaceutical composition with colonic delivery obtained at the end of the process according to the invention is in the form of microgranules.

According to a preferred embodiment, the anionic (meth)acrylate copolymer that is soluble at a pH greater than 5.5 of step a) or a′) is a methacrylic acid-ethyl acrylate (1:1) copolymer. Preferably, said methacrylic acid-ethyl acrylate (1:1) copolymer used is the marketed compound Eudragit® L30D. The choice of this copolymer as a binder is due to its interest in the dusting technique.

According to a preferred embodiment, among the copolymers of step d) said anionic (meth)acrylate copolymer that is soluble at a pH greater than 6 is a methacrylic acid-methyl methacrylate (1:1) copolymer. Preferably, said methacrylic acid-methyl methacrylate (1:1) copolymer used is the marketed compound Eudragit® L100. This copolymer allows to delay the release in the intestinal tract of the compounds coated by this copolymer until reaching the jejunum.

According to a preferred embodiment, among the copolymers of step d) said anionic (meth)acrylate copolymer that is soluble at a pH greater than 7 is a methacrylic acid-methyl methacrylate (1:2) copolymer. Preferably, said methacrylic acid-methyl methacrylate (1:2) copolymer used is the marketed compound Eudragit® 5100. This copolymer allows to delay the release in the intestinal tract of the compounds coated by this copolymer until reaching the ileum and the colon.

According to a preferred embodiment, among the copolymers of step d) said copolymer that is insoluble in an aqueous medium is an ethyl acrylate-methyl methacrylate-methacrylic acid ester with a quaternary ammonium group copolymer (1:2:0.2), advantageously an ethyl acrylate-methyl methacrylate-methacrylic acid ester with a trimethylammonioethyl methacrylate chloride group copolymer (1:2:0.2). Preferably, said ethyl acrylate-methyl methacrylate-methacrylic acid ester with quaternary ammonium group copolymer used is the marketed compound Eudragit® RL100. This copolymer allows to control over time the release of the compounds coated with this copolymer.

According to a preferred embodiment, the composition sprayed in step d) has a ratio anionic (meth)acrylate copolymer that is soluble at a pH greater than 6: anionic (meth)acrylate copolymer that is soluble at a pH greater than 7: anionic (meth)acrylate copolymer that is insoluble in an aqueous medium of 4:3:3. The mixture of the three copolymers of the coating allows to obtain a release of the pharmaceutical composition which differs according to the pH because these three polymers dissolve at different pHs. This mixture of the three polymers thus allows the generation of asperities of the coating as the pharmaceutical composition moves in the intestinal transit and the release of the product at the desired place, that is to say in the colon.

In the context of the present invention, the three polymers of the coating are applied in the same layer. This differs from prior art coatings in which each polymer forms a separate layer. Thus, the pharmaceutical composition prepared according to the process according to the invention may comprise one or more coating layer(s), each of the layers comprising the mixture of the three polymers mentioned above.

Advantageously, the pharmaceutical composition prepared according to the process of the invention comprises a single coating layer.

The preparation of a pharmaceutical composition with a coating comprising all the polymers in a single layer and advantageously comprising a coating monolayer, in particular allows a significant time saving and therefore an economic gain. Furthermore, the monolayer coating allows better adjustment of the coating rate, in particular in the event of a technical problem during its preparation (compressed air problem, nozzle blockage, or other). Indeed, when the coating is multilayer, each of the layers comprising a different polymer, an adjustment of the coating and in particular of the first coating layers cannot be carried out, which is problematic, in particular if a technical problem occurs during application of the first layers. Indeed, technical problems are only detected at the end of the process, that is to say when all the layers have been applied, so it will not be possible to readjust the coating of the first layers.

According to a preferred embodiment, the dusting of the active ingredient in step b′) is carried out by manual or mechanical dusting in at least one conventional flat-bottomed turbine. This manual or mechanical dusting process allows to obtain and guarantee, advantageously, cores of the pharmaceutical composition in the form of microgranules having a narrow particle size distribution.

This narrow particle size subsequently allows to produce a uniform and reproducible coating around the microgranules; indeed, as the microgranules all have an equivalent size thanks to manual or mechanical dusting, they will receive the same amount of polymer which will form a layer of equivalent thickness on all the microgranules. During the administration of the pharmaceutical composition, this will allow to obtain low coefficients of variation during the dissolution of the granules in the body, that is to say a uniform dissolution between the microgranules coated with the pharmaceutical composition.

Advantageously and in a non-limiting manner, the active ingredient intended to be delivered in the colon can be an anti-infective, for example an antibiotic, an anti-inflammatory, anti-histamine, anti-cholinergic, antiviral, antimitotic, peptides, proteins, genes, antisense oligonucleotides, diagnostic agents and/or immunosuppressive agents or bacteria. Advantageously, the active ingredient intended to be delivered in the colon can be water-soluble or fat-soluble.

Among the active ingredients intended to be delivered in the colon that are particularly advantageous, there are anti-inflammatory agents, antitumor agents, antisense oligonucleotides and enzymes capable of inactivating antibiotics in the colon, in particular β-lactamases or enzymes capable of inactivating macrolides and relatives such as erythromycin esterase.

More advantageously, the active ingredient intended to be delivered in the colon is an anti-inflammatory agent.

Preferably, the active ingredient intended to be delivered in the colon is selected from salicylazosulfapyridine or sulfasalazine (Salazopyrine), 5-aminosalicylic acid (mesalazine), budesonide, rifamycin, acamprosate or linaclotide.

### FIGURES

FIG. 1: Describes the results of Example 3.

The following examples are intended to illustrate the present invention in a non-limiting manner.

### EXAMPLES

### Example 1: Preparation of a Pharmaceutical Composition According to the Invention by Dusting in a Conventional Turbine

Composition A described in Table 2 was prepared according to the following protocol:

1) Assembling Mesalazine Microgranules:
Introducing neutral supports in the turbine (Mass temperature; 15-35° C.);
Spraying the binding suspension comprising Eudragit L30D and triethylcitrate;
Dusting the active ingredient by manual or mechanical dusting;
Drying; and
Sieving on a vibrating sieve of the SODEVA type with grids from 500 to 1000 μm.

At the end of assembly, the microgranules have a D50 comprised between 500 and 700 μm and a D90 comprised between 700 and 950 μm.

2) Coating

Following the assembly step, the microgranules are coated by spraying the coating suspension on the active microgranules.

Composition of the coating suspension for a weight gain of 10% in polymer for a spray on 1000.0 g of active grains of Mesalazine:
Eudragit 5100: 30.0 g
Eudragit RL100: 30.0 g
Eudragit L100: 40.0 g
TEC (triethylcitrate): 15.0 g
Talc: 40.0 g
Ethanol: 1395.0 g

The coating suspension is prepared according to the following protocol:
Weigh the Ethanol in a container of adapted capacity and stir it with a stirrer (IKA type) equipped with a propeller.
Incorporate Eudragit® 5100 with stirring and wait for complete dissolution.
Incorporate Eudragit® L100 with stirring and wait for complete dissolution.
Incorporate Eudragit® RL100 with stirring and wait for complete dissolution.
Add the Triethyl Citrate with stirring and maintain stirring for 1 hour.
Incorporate the Talc while stirring and wait 30 minutes before starting the spraying.
Spray the coating suspension onto the microgranules while maintaining agitation throughout the spraying, as described below.

The coating is carried out in a Glatt GPCG1 type fluidized air bed in Wurster mode equipped with a Schlick gun (1.2 mm nozzle).

Coating Parameters:

TABLE 1

| Parameters | Setpoint | |
|---|---|---|
| Air inlet temperature | 30-35° | C. |
| Product temperature | 25-35° | C. |
| Air flow rate | 40-60 | m³/h |
| Suspension spray flow rate | 3-10 | g/min |
| Nebulization air | 1-2 | bar |

At the end of the coating, the microgranules are sieved on a grid of adapted size.

At the end of the coating, the microgranules have a D50 comprised between 600 and 800 μm and a D90 comprised between 800 and 950 μm.

TABLE 2

| Composition A according | | Sachets of | | Sachets of | |
|---|---|---|---|---|---|
| to the invention | | 1000 mg | | 2000 mg | |
| Active pharmaceutical | Mesalazine | 1000 | mg | 2000 | mg |
| ingredient | | | | | |
| Neutral supports | Neutral SP | 594.4 | mg | 1188.8 | mg |
| | 400-500 μm | | | | |
| Anionic (meth)acrylate | Eudragit ® | 74.9 | mg | 149.7 | mg |
| copolymer that | L30D | | | | |
| is soluble at a pH | | | | | |
| greater than 5.5 | | | | | |
| Anionic (meth)acrylate | Eudragit ® | 67.1 | mg | 134.1 | mg |
| copolymer that | L100 | | | | |
| is soluble at a pH | | | | | |
| greater than 6 | | | | | |
| Anionic (meth)acrylate | Eudragit ® | 50.3 | mg | 100.6 | mg |
| copolymer that | S100 | | | | |
| is soluble at a pH | | | | | |
| greater than 7 | | | | | |
| Water-insoluble anionic | Eudragit ® | 50.3 | mg | 100.6 | mg |
| (meth)acrylate copolymer | RL100 | | | | |
| Plasticizer | Triethyl Citrate | 25.2 | mg | 50.3 | mg |
| Lubricant | Talc | 67.1 | mg | 134.1 | mg |

| | | | | |
|---|---|---|---|---|
| Total | 1936.6 | mg | 3873.2 | mg |

### Comparative Example 2: Preparation of Comparative Compositions B and C by Dusting in a Conventional Turbine

Comparative compositions B and C described in Tables 3 and 4 were prepared according to the same protocol as that described in example 1.

TABLE 3
Comparative composition B

| | | |
|---|---|---|
| Core | Mesalazine | 52.8% |
| | Neutral SP 400-500 μm | 31.4% |
| | Eudragit ® L30D | 4.0% |
| | Triethylcitrate | 0.4% |
| Coating 1 | Eudragit ® E100 | 2.7% |
| | Triethylcitrate | 0.3% |
| | Talc | 1.3% |
| Coating 2 | Eudragit ® S100 | 4.6% |
| | Triethylcitrate | 0.7% |
| | Talc | 1.8% |
| | | 100.0% |

TABLE 4
Comparative composition C

| | | |
|---|---|---|
| Core | Mesalazine | 51.6% |
| | Neutral SP 400-500 μm | 30.7% |
| | Eudragit ® L30D | 3.9% |
| | Triethylcitrate | 0.4% |
| Coating | Eudragit ® S100 | 8.7% |
| | Triethylcitrate | 1.3% |
| | Talc | 3.4% |

| | |
|---|---|
| TOTAL | 100.0% |

### Example 3: Study of the Dissolution Profile of Compositions A, B and C

Protocol: the apparatus used is a USP II type dissolutest (stirring with blades). The equivalent of 1 g of active ingredient in the form of microgranules is introduced into a vessel containing 750 mL of 0.1N HCl, stirred for 2 hours at a speed of 50 rpm. A sample of 10 mL is taken after 2 hours. The grains are recovered and introduced into a vessel containing 950 mL of a buffer medium of pH 6.8 (composed of 6.805 g of KH₂PO₄ and 22.4 mL of NaOH per 1 L) stirred for 1 hour at a speed of 50 rpm. A sample of 10 mL is taken after 1 hour. 50 mL of a 0.36N NaOH solution are added to the vessel. Agitation is activated at 50 rpm. 5 mL samples are taken after 30, 45, 60, 90 and 120 min.

The samples are then analyzed by liquid chromatography and the amount of active ingredient released is determined by UV detection.

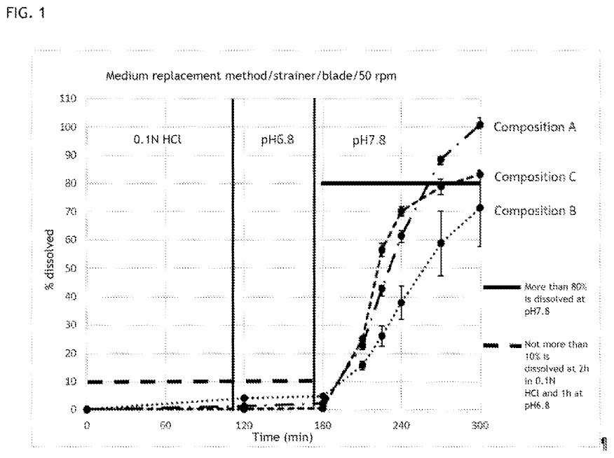

Results: the results are presented in FIG. 1, which illustrates the percentages of dissolution of compositions A, B and C as a function of time.

Conclusion: the dissolution profile of composition A is: <10% after 2 hours in 0.1N HCl and 1 hour at pH6.8, then >80% after 2 hours in pH7.8.

The inventors have thus demonstrated that the process according to the invention allows to obtain a pharmaceutical composition having a uniform and reproducible dissolution and therefore likewise uniform and reproducible release of the active ingredient with low coefficients of variation.

1. A process for preparing an orally administered pharmaceutical composition with colonic delivery comprising at least one core and a coating layer, characterized in that it comprises the following steps:
a) Spraying onto a neutral support an aqueous suspension comprising at least one anionic (meth)acrylate copolymer that is soluble at a pH greater than 5.5 and at least one active ingredient intended to be delivered in the colon;
or
a′) Spraying onto a neutral support an aqueous suspension comprising at least one anionic (meth)acrylate copolymer that is soluble at a pH greater than 5.5 then
b′) Dusting at least one active ingredient intended to be delivered in the colon onto the microgranules obtained after step a′);
c′) carrying out steps a′) and b′) alternately until the desired content of active ingredient has been obtained and
d) Coating the microgranules obtained after step a) or c′) by spraying a composition comprising at least one anionic (meth)acrylate copolymer that is soluble at a pH greater than 6, an anionic (meth)acrylate copolymer that is soluble at a pH greater than 7 and an anionic (meth)acrylate copolymer that is insoluble in an aqueous medium.

2. The process according to claim 1, characterized in that said anionic (meth)acrylate copolymer that is soluble at a pH greater than 5.5 of step a) or a′) is a methacrylic acid-ethyl acrylate (1:1) copolymer.

3. The process according to claim 1 or 2, characterized in that among the copolymers of step d) said anionic (meth)acrylate copolymer that is soluble at a pH greater than 6 is a methacrylic acid-methyl methacrylate (1:1) copolymer.

4. The process according to any one of claims 1 to 3, characterized in that among the copolymers of step d) said anionic (meth)acrylate copolymer that is soluble at a pH greater than 7 is a methacrylic acid-methyl methacrylate (1:2) copolymer.

5. The process according to any one of claims 1 to 4, characterized in that among the copolymers of step d) said copolymer that is insoluble in an aqueous medium is an ethyl acrylate-methyl methacrylate-methacrylic acid ester with a quaternary ammonium group copolymer (1:2:0.2), advantageously an ethyl acrylate-methyl methacrylate-methacrylic acid ester with a trimethylammonioethyl methacrylate chloride group copolymer (1:2:0.2).

6. The process according to any one of claims 1 to 5, characterized in that the composition sprayed in step d) has a ratio anionic (meth)acrylate copolymer that is soluble at a pH greater than 6: anionic (meth)acrylate copolymer that is soluble at a pH greater than 7: anionic (meth)acrylate copolymer that is insoluble in an aqueous medium of 4:3:3.

7. The process according to any one of claims 1 to 6, characterized in that said active ingredient intended to be delivered in the colon is selected from sulfasalazine, 5-aminosalicylic acid (mesalazine), budesonide, rifamycin, acamprosate or linaclotide.

8. The process according to any one of claims 1 to 7, characterized in that the orally administered pharmaceutical composition with colonic delivery obtained at the end of the process is in the form of microgranules.

9. The process according to any one of claims 1 to 8, characterized in that the dusting of the active ingredient in step b′) is carried out by manual or mechanical dusting in at least one conventional turbine.
